SEQUENCE LISTING (2 sequences)

SEQ ID NO: 1 - protein, 6 aa, synthetic construct
EEMQRR

SEQ ID NO: 2 - protein, 13 aa, synthetic construct
ELEEMQRRADQLA

(19) United States
(12) Patent Application Publication
(10) Pub. No.: US 2025/0161409 A1
(43) Pub. Date: May 22, 2025

(54) COMPOSITIONS FOR TREATING HYPERHIDROSIS

(71) Applicant: PROSPERA BIOTECH, S.L., Elche (ES)

(72) Inventors: Antonio Vicente FERRER MONTIEL, Playa De San Juan (ES); Clotilde FERRÁNDIZ HUERTAS, Elche (ES); Marta GARCÍA ESCOLANO, Campello (ES)

(73) Assignee: PROSPERA BIOTECH, S.L., Elche (ES)

(21) Appl. No.: 18/841,333

(22) PCT Filed: Feb. 22, 2023
(86) PCT No.: PCT/EP2023/054396

§ 371 Date: Aug. 23, 2024

(30) Foreign Application Priority Data
Feb. 23, 2022 (EP) 22382149.7

(51) Int. Cl. A61K 38/17; A61K 31/165; A61P 17/00

(52) U.S. Cl. CPC A61K 38/177; A61K 31/165; A61P 17/00

(57) ABSTRACT

The present disclosure relates to the use of a combination of a known neuronal exocytosis inhibitor, and a known TRPV1 modulator in the treatment and/or prevention of diseases or conditions that courses through an excessive neural stimulation of the eccrine gland, in particular hyperhidrosis or excessive sweat. It also relates to these combinations, and to pharmaceutical and cosmetic compositions containing them.

This application claims the benefit of European Patent Application EP22382149.7 filed on Feb. 23, 2022.

### TECHNICAL FIELD

The present disclosure relates to the field of pharmacy and cosmetics, in particular it relates to the use of a combination of known active agents, being one of them a known neuronal exocytosis inhibitor, and the other a known TRPV1 modulator, in the treatment and/or prevention of diseases or conditions that courses through an excessive neural stimulation of the eccrine gland, in particular hyperhidrosis. It also relates to these combinations, and to pharmaceutical and cosmetic compositions containing them.

### BACKGROUND

Sweating is a physiological function necessary for the regulation of body temperature when the ambient temperature is high. Sweating cools the body and blood through evaporation of water contained in sweat. When there is a high environmental temperature and/or when muscular heat is generated by physical exercise, the neurosensory system sends a signal to the brain to stimulate the cutaneous sweat glands and produce a vasodilatation that allows cooling, maintaining the body's thermal homeostasis. This phenomenon can also occur due to an efferent action of the brain in certain emotional or stressful situations. This physiological activity is reversible so that sweating stops when the triggering stimulus disappears. The interdependence between the cutaneous thermosensory system and the eccrine system ensures correct control of body temperature. An alteration either in the threshold of detection of environmental temperatures or an overexcitation of the eccrine glands often leads to states of excessive sweating that can evolve over time to hyperhidrosis syndrome, characterized by a dysregulation of the eccrine glands that produce virtually continuous sweating.

Primary hyperhidrosis is an idiopathic, focal, bilateral, symmetrical, exaggerated sweating that typically affects the axillae, palms, soles, and craniofacial regions and is not caused by any underlying medical disease or medication. Primary hyperhidrosis is diagnosed in 93% of all patients with excessive sweating. Currently, the therapeutic approach to controlling excessive sweating is based on the use of aluminum salts which primarily have a drying effect, although they can also reduce eccrine secretion. Aluminum salts, mainly aluminum hydroxide, may have estrogenic activity in women, and their use has raised concern and is currently questioned.

Acetycholine is the major neurotransmitter that activates the eccrine gland, thus mediates sweat secretion. Therefore, drugs that inhibit the binding of acetylcholine, called anticholinergic agents, can be used to treat hyperhidrosis. Neuromuscular blocking drugs such as botulinum toxin-A can treat hyperhidrosis by inhibiting synaptic vesicle fusion and release of acetylcholine into the synaptic cleft which prevents acetylcholine action at the post-synaptic receptors and prevents sweating. The injection of botulin toxin is quite active, but needs to be administered by a physician, is very painful requiring the use of anesthetics and has a limited duration. As an alternative to the use of botulinum toxin, the use of small peptides that interfere with the release of acetylcholine in the neuro-eccrine terminal was proposed.

SNARE (soluble N-ethylmaleimide-sensitive factor attachment protein receptor; SNAP receptor) proteins are a large protein superfamily consisting of 60 or more members in yeasts and mammalian cells, and the primary role of SNARE proteins is to mediate the fusion of vesicles with their target membrane bound to compartments.

The neuronal protein SNAP-25 (Synaptosomal-Associated Protein, 25 kDa) plays a key role in neurosecretion since it is involved in the formation of the SNARE complex, which manages and controls the release of acetylcholine accumulated in vesicles, thus it constitutes a key target for controlling neurosecretion. The truncation of any of the proteins which form the fusion complex prevents their assembling and, therefore, inhibits vesicle release and inhibits neuronal exocytosis. The nucleus of this fusion complex is comprised of the proteins SNAP-25 and syntaxin, located in the presynaptic plasma membrane, and the synaptobrevin protein of the VAMP family of proteins, located in the vesicular plasma membrane.

It is known in the prior art that certain peptides derived from the protein sequences which form the SNARE complex are capable of inhibiting neuronal exocytosis, such as peptides derived from the amino and carboxy-terminal domains of the protein SNAP-25, the peptides derived from the sequence of syntaxin amino acids, of the synaptobrevin, of the synaptotagmin, and of the protein snapin. Similarly, synthetic peptides obtained by rational design or by searching synthetic libraries which are capable of inhibiting neuronal exocytosis by interfering in the formation of the SNARE complex have also been described.

EP1180524 describes peptides derived from the amino terminal fragment of the protein SNAP-25 which have anti-wrinkle effects. In particular, in this patent document it is described an acetyl-hexapeptide, commercialized under the tradename Argireline® by Lipotec, S. A. (Ac-Glu-Glu-Met-Gln-Arg-Arg; CAS Reg No. 616204-22-9; (4S)-4-acetamido-5-[[(2S)-1-[[(2S)-1-[[(2S)-5-amino-1-[[(2S)-1-[[(2S)-1-amino-5-(diaminomethylideneamino)-1-oxopentan-2-yl]amino]-5-(diaminomethylideneamino)-1-oxopentan-2-yl]amino]-1,5-dioxopentan-2-yl]amino]-4-methylsulfanyl-1-oxobutan-2-yl]amino]-4-carboxy-1-oxobutan-2-yl]amino]-5-oxopentanoic acid, acetic acid; see SEQ. ID NO. 1).

EP1856139 describes a composition comprising Argireline® and a positively charged carrier comprising a positively charged polylysine, with attached positively charged branching groups independently selected from -(gly) n1-(arg) n2, HIV-TAT, Antennapedia PTD, and fragments of HIV-TAT or of Antennapedia PTD or mixtures thereof, in which the subscript n1 is an integer of from 0 to 20, and the subscript n2 is independently an odd integer of from 5 to 25, wherein the oligopeptide is not covalently modified by the positively charged carrier. The composition is used in a method of treating or reducing the symptoms associated with hyperhydrosis. Formulations containing at least 10 wt % were necessary in order to obtain a noticeable result.

The transient receptor potential cation channel subfamily V member 1 (TRPV1), also known as the capsaicin receptor and the vanilloid receptor 1, is a protein that, in humans, is encoded by the TRPV1 gene. It was the first isolated member of the transient receptor potential vanilloid receptor proteins that in turn are a sub-family of the transient receptor potential protein group. This protein is a member of the TRPV group of transient receptor potential family of ion channels. TRPV1 is an element of or mechanism used by the mammalian somatosensory system. It is a nonselective cation channel that may be activated by a wide variety of exogenous and endogenous physical and chemical stimuli. The best-known activators of TRPV1 are: temperature greater than 43° C. (109° F.); acidic conditions; capsaicin (the irritating compound in hot chili peppers); and allyl isothiocyanate.

EP3621950 describes modulators of TRPV1 having a 3,4-dioxy substituted phenyl moiety linked to a substituted (acetyloxy)acetamidoalkyl moiety. In particular, in this patent document it is described 2-((4-hydroxy-2-iodo-5-methoxybenzyl)amino)-2-oxoethyl nonanoate, commercialized under the tradename Calmapsin® by Antalgenics, S.L. According to EP3621950, the TRPV1 modulators are useful in the treatment and/or prevention of pain, inflammation, cancer, and other diseases such as sensitive skin, itch (pruritus), rosacea, acne vulgaris, atopic dermatitis, psoriasis and psoriatic arthritis.

From what is known in the art it is derived that there is still the need of providing more effective compositions for the therapeutical and non-therapeutical, cosmetic, treatment and/or prevention of hyperhidrosis.

### SUMMARY

Inventors have found that the combination of a known neuronal exocytosis inhibitor, and a known TRPV1 modulator results in a synergic effect in the therapeutical and non-therapeutical, cosmetic, treatment and/or prevention of hyperhidrosis.

Excessive sweating, or sweating in general, can constitute a pathological condition in the sense of hyperhidrosis, such pathological condition may require therapeutic treatment. However, excessive sweating can also constitute a mere cosmetic, non-therapeutic, problem. Both, therapeutic and cosmetic uses are addressed in the present invention.

Argireline®, an acetylated derivate of SEQ. ID NO. 1, a 6 amino acid peptide derived from the N-terminal end of the SNAP-25 protein of the SNARE neuronal exocytosis complex, has been described as an alternative to the use of botulinum toxin. This peptide is a modest inhibitor of calcium-regulated neuronal exocytosis. A formulation containing 0.5-1.0% Argireline® has a modest effect in attenuating hyperhidrosis and excessive sweating, indicating that it was not sufficiently effective in reducing eccrine gland activity.

Similarly, the use of a TRPV1 thermoreceptor blocker, 2-((4-hydroxy-2-iodo-5-methoxybenzyl)amino)-2-oxoethyl nonanoate (Calmpasin®) (0.1-0.01%), was also unable to attenuate excessive sweating.

Surprisingly, however, the combination of both products, the acetylated derivative of SEQ. ID NO. 1 (Argireline®) (0.05%) and 2-((4-hydroxy-2-iodo-5-methoxybenzyl)amino)-2-oxoethyl nonanoate (Calmpasin®) (0.01%) resulted in a significant attenuation of excessive sweating, indicating a synergistic effect in the combination of both actives, or, more generally, of acting on both thermosensory and neuro-eccrine systems.

A person skilled in the art could not deduce the existence of a synergism in the anti-hyperhidrosis effect when neuronal exocytosis inhibitors and TRPV1 modulators are combined since the mechanism of action of the active ingredients is different.

The regulation of sweat production occurs predominantly through neurohormonal mechanisms, for which the hypothalamic preoptic area plays a vital role. An increase in temperature in this region through an increase in core temperature provides the strongest stimulus for sweat production. The preoptic hypothalamic area can also be activated when cutaneous C-fibers in a localized area of the skin are activated by an increase in ambient temperature.

While not wishing to be being bound by theory, the inventors have hypothesized that the etiology of hyperhidrosis may be due to a complicated malfunction of the autonomic nervous system involving both the sympathetic and parasympathetic systems, causing neurogenic hyperexcitability of reflex circuits, leading to hyperstimulation of the normal eccrine sweat glands.

A change in external temperature is initially detected by primary afferent nerve fibers of the somatosensory system in the skin. Transient receptor potential (TRP) ion channels function as molecular sensors of both physical (thermal and mechanical) and chemical environmental stimuli. Transient receptor potential receptor vanilloid 1 (TRPV1) is a polymodal receptor that is activated at temperatures >40° C., as well as by molecules such as capsaicin, the spicy ingredient in chili peppers. The TRPV1 receptor is a cation-permeable ion channel that contributes to the firing of electrical impulses in the somatosensory system, indicating the presence of high temperatures and/or the presence of compounds such as capsaicin. In both cases, TRPV1 receptor activation is usually accompanied by vasodilation and, depending on the intensity of the stimulus and its duration, by the process of sweating. In addition, TRPV1 receptor activity is enhanced by pro-inflammatory mediators and an acidic pH. Potentiation or sensitization of the TRPV1 receptor may be due to a decrease in its thermal activation threshold (<40° C.) and/or an increase of receptors in the thermosensory terminal. In both cases, the result is an increase in receptor activity whose signaling intensity is interpreted by the brain as an increase in environmental temperature. As a result, the brain activates cooling mechanisms, i.e. vasodilation and sweating.

Therefore, and without being bound by theory, the inventors hypothesize that excessive sweating, particularly hyperhidrosis, may be caused by dysregulation of the thermosensory system contributing to an overstimulation of the eccrine gland. This molecular mechanism suggests that simultaneous attenuation of the two systems, thermosensory and eccrine, may exert a synergistic action allowing better control of excessive local sweating.

The inventors have found that the use of a known TRPV1 modulator, when is combined with a known neuronal exocytosis inhibitor result in a synergistic effect in the attenuation of excessive sweating, bringing it to a physiological level, in a safe and satisfactory way for those affected. More particularly, in accordance with some particular embodiments, when a modest blocker of the TRPV1 thermoreceptor, such as those described in EP3621950, more particularly 2-((4-hydroxy-2-iodo-5-methoxybenzyl)amino)-2-oxoethyl nonanoate, Calmapsin®, which allows restoring its activation at T>40°, together with a known moderate inhibitor of acetylcholine release in the neurons innervating the eccrine gland, i.e. a known neuronal exocytosis inhibitor, such as those described in EP1180524, more particularly a peptide of SEQ. ID. NO 2, or an acetylated form either at the N-terminus or at the C-terminus, more particularly Argireline®, results in the attenuation of excessive sweating, bringing it to a physiological level, in a safe and satisfactory way for those affected.

As shown in the experimental section, the results obtained for the combination of 2-((4-hydroxy-2-iodo-5-methoxybenzyl)amino)-2-oxoethyl nonanoate and an acetylated derivative of the peptide of SEQ. ID NO. 1, suggest that a combination of a known neuronal exocytosis inhibitor and a known TRPV1 modulator promotes a higher combined action that the one that could be expected on the performance of the agents when used alone, due to a synergistic effect. From the results of the experimental data herein disclosed, it may be concluded that synergy has been demonstrated since the performance of the combination of a known neuronal exocytosis inhibitor and a known TRPV1 modulator is indeed better than could justificably have been predicted.

Thus, a first aspect of the present invention refers to a composition comprising a synergistic combination of a known neuronal exocytosis inhibitor; and a known TRPV1 modulator.

A second aspect of the present invention refers to a pharmaceutical or cosmetic composition which comprises an effective amount of the composition as defined herein together with one or more pharmaceutically or cosmetically acceptable excipients or carriers.

A third aspect of the present invention refers to a known TRPV1 modulator selected from the group of known TRPV1 agonists capable of desensitizing TRPV1 with an EC50 value≤10 μM, preferably an EC50 value≤8 μM, more preferably ≤1 μM, more preferably ≤500 nM and known TRPV1 antagonists capable of inhibiting TRPV1 with an IC50 value≤100 μM, preferably an IC50≤75 μM, more preferably IC50≤50 μM, more preferably IC50≤30 μM, more preferably IC50≤10 μM; where the activation/inhibition of TRPV1 is measured by calcium microfluorometry assays as described herein below, together with one or more pharmaceutically acceptable excipients or carriers, for administration in combination with a known neuronal exocytosis inhibitor selected from those which at a concentration of 1 mM inhibit from 15% to 45% of the release of catecholamines in the permeabilized chromaffin cells, preferably from 17% to 40%, more preferably from 20% to 35%; where the inhibition of neural-exocytosis is measured by evaluating the strength of said compounds in inhibiting the release of catecholamines induced by calcium in chromaffin cells permeabilized with the detergent digitonin, in accordance with the method described by Gutiérrez et al. (FEBS Lett 1995, 372, 39-43, and J. Biol. Chem. 1997, 272, 2634-2639), or those which at a concentration of 0.1 mM inhibit from 25% to 50% of the release of α-calcitonin gene-related peptide (α-CGRP) in intact sensory neurons, preferably from 25% to 45%, more preferably from 25% to 40%, where the inhibition of neural-exocytosis is measured by enzyme immunoassay evaluating the strength of said compounds in inhibiting the release of α-CGRP from intact primary sensory neurons in accordance with the method described by Butrón et al. (Bioorg Chem. 2021 October; 115:105231. doi:10.1016/j.bioorg.2021.105231); for simultaneous, concurrent, or sequential use in the treatment and/or prevention of hyperhidrosis.

A fourth aspect of the present invention refers to the pharmaceutical composition as defined herein, for use in the prevention and/or treatment of a disease or condition which courses through an excessive neural stimulation of the eccrine gland, wherein the disease or condition is hyperhidrosis.

A fifth aspect of the present invention refers to the combination or the cosmetic composition as defined herein for the cosmetic, non-therapeutically treatment of perspiration or excessive sweating, i.e. for the skincare of the areas with excessive sweating.

An additional aspect of the present invention refers to a method of cosmetic, non-therapeutic treatment of a subject comprising administering a cosmetically effective amount of a combination or a cosmetic composition as defined herein to a subject, wherein the cosmetic, non-therapeutic treatment is the treatment and/or prevention of perspiration.

In another aspect of the present invention, it is provided a cosmetic method for the treatment of human excessive perspiration comprising the use of the combination or a cosmetic composition as defined herein.

Additionally, an aspect of the present invention refers to the use of the cosmetic composition as defined above as a skin care agent, where the skin care comprises ameliorating excessive perspiration.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “about” or “around” as used herein refers to a range of values ±10% of a specified value. For example, the expression “about 10” or “around 10” includes ±10% of 10, i.e. from 9 to 11.

The term “weight ratio” refers to the relation of weights of the known neuronal exocytosis inhibitor to the known TRPV1 modulator.

The term “percentage (%) by weight” refers to the percentage of each ingredient of the combination or composition in relation to the total weight.

An “effective amount” of the combination refers to the amount of active ingredients which provide a therapeutic or cosmetic effect after its application. Therefore, the expression “therapeutically or cosmetically effective amount” as used herein refers to the amount of the composition that, when administered, is sufficient to prevent development of, or alleviate to some extent, one or more of the symptoms of the disease or condition which is addressed.

As used herein, unless otherwise stated, the term “treating” and “treatment” in relation to a disease, disorder or condition refers to the management and care of a patient for the purpose of combating a disease, disorder or condition, such as to reverse, alleviate, inhibit the process of, or prevent the disease, disorder or condition to which such term applies, or one or more symptoms of such disease, disorder or condition, and includes the administration of a composition of the invention to prevent the onset of the symptoms or the complications, or alleviating the symptoms or complications, or eliminating the disease, condition or disorder. Preferably, treatment is curative or ameliorating.

The term “treatment”, as used in the context of this specification when it is not accompanied by the qualifications “cosmetic” and/or “non-therapeutic”, means the administration of a compound according to the invention to alleviate or eliminate a disease or disorder or reduce or eliminate one or more symptoms associated with this disease or disorder. The term “treatment” also covers the ability to alleviate or eliminate the physiological consequences of the disease or disorder.

When the term “treatment” is accompanied by the qualifications “cosmetic, non-therapeutic” they refer to the application of the compound to the skin, hair and/or in particular with the aim of improving the cosmetic qualities of the skin, and/or hair such as and not restricted to, their level of hydration, elasticity, firmness, shine, tone or texture, among others. The term “care” in this invention refers to the maintenance of the qualities of the skin, and/or hair. These qualities are subject to improvement and maintained through a cosmetic treatment and/or care of the skin, and/or hair both in healthy subjects as well as those which present diseases and/or disorders of the skin, such as and not restricted to, ulcers and lesions on the skin, psoriasis, dermatitis, acne or rosacea, among others.

The term “prevention”, as used in this invention, refers to the ability of a compound of the invention to prevent, delay or hinder the appearance or development of a disease or disorder before its appearance.

For the purposes of the invention, the term “treatment” of the disease refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

Unless otherwise indicated, in the context of the present invention the percentage and amount of a certain component in a composition are to be referred to the weight of said component with respect to the total weight of the composition.

Unless otherwise stated, all percentages mentioned herein regarding the components of the composition are expressed in weight with respect to the total weight of the composition, provided that the sum of the amounts of the components is equal to 100%.

For the purposes of the invention, a carrier is suitable for use in the pharmaceutical compositions described herein if it is compatible with the other ingredients of the composition and not deleterious to the recipient of the composition. The expression “pharmaceutically or cosmetically acceptable diluents, excipients or carriers” refers to pharmaceutically or cosmetically acceptable materials, compositions or vehicles suitable for use in the pharmaceutical or cosmetic technology for preparing compositions with medical (therapeutic) and non-medical (non-therapeutic) use. Each component must be pharmaceutically or cosmetically acceptable in the sense of being compatible with the other ingredients of the pharmaceutical or cosmetical composition. It must also be suitable for use in contact with the tissue or organ of humans without excessive toxicity, irritation, allergic response, immunogenicity or other problems or complications commensurate with a reasonable benefit/risk ratio. A “pharmaceutically or cosmetically acceptable carrier” includes non-API (API refers to Active Pharmaceutical Ingredient) substances, such as disintegrators, binders, fillers, lubricants and the like, used in formulating pharmaceutical or cosmetical products and regarded as safe for administering to subjects (particularly humans) according to established governmental standards, including those promulgated by the United States Food and Drug Administration and the European Medical Agency. Pharmaceutically and cosmetically acceptable carriers are well known to those skilled in the art and are selected on the basis of the chosen type of formulation and route of administration, according to standard pharmaceutical or cosmetical practice. Unless otherwise indicated, in the context of the present invention a range of values indicated for a certain parameter, for example the weight of a component in a mixture, includes the upper and the lower limits of the range, e.g. if the content in weight, or in volume, of a component A in a mixture is indicated as “X to Y”, the content of A can be X, Y or any of the intermediate values.

With regard to the specific conditions for carrying out the preparation process of the invention, the skilled person would know how to adjust the parameters of each of the steps indicated above in the light of the description and examples of the present invention.

As mentioned above, a first aspect of the invention relates to a composition comprising a synergistic combination of:
a) a known neuronal exocytosis inhibitor; and
b) a known TRPV1 modulator.

In one embodiment, optionally in combination with one or more features of the various embodiments described above or below, the known TRPV1 modulator is selected from the group consisting of a TRPV1 agonist capable of desensitizing TRPV1 with an EC50 value≤10 μM, preferably an EC50 value≤8, more preferably ≤1 μM, more preferably ≤500 nM and a known TRPV1 antagonist capable of inhibiting TRPV1 with an IC50 value≤100 μM, preferably an IC50≤75 μM, more preferably IC50≤50 μM, more preferably IC50≤30 μM, more preferably IC50≤10 μM, where the activation/inhibition of TRPV1 is determined according to the method identified below.

Briefly, the activity of the compounds was determined on TRPV1 expressing cells (TRPV1-SH-SY5Y) by Calcium microfluorometry assay. SH-SY5Y cells stably expressing rat TRPV1 channel (SH-SY5Y-TRPV1) were grown in Earle's minimum essential medium (MEM) containing 10% (v/v) of FCS, 1% nonessential amino acids, 2 mM L-glutamine, 100 μg/ml streptomycin, 100 U/ml penicillin, and 0.4 μg/ml puromycin in a humidity-controlled incubator with 5% CO₂ and at 37° C. For fluorescence assays, cells expressing TRPV1 channel (TRPV1-SH-SY5Y) were seeded in 96-well plates (Corning Incorporated, Corning, NY) at a cell density of 40.000 cells 2 days before treatment. The day of treatment the medium was replaced with 100 μL of the dye loading solution Fluo-4 NW supplemented with probenecid 2.5 mM. After incubation at 37° C. in a humidified atmosphere of 5% CO₂ for 60 minutes, plates were transferred to a fluorescence plate reader (Polastar Omega BMG Labtech). The baseline fluorescence of Fluo-4 dye (emission 485 nm/excitation 520 nm) was recorded for 4 cycles. Then, 1 μL of vehicle (DMSO) or compounds at 10 μM final concentration were added to the well, and fluorescence intensity was recorded during 10 cycles more prior to stimulation with the agonist (10 μM capsaicin for TRPV1). The antagonist (10 μM Ruthenium Red for TRPV1) was added for the blockade. The changes in fluorescence intensity were recorded during 10 cycles more.

Additionally, The IC50 (half-maximum inhibitory concentration) value is the concentration at which a given compound inhibits 50% of the channel activity, in this case TRPV1, and it can serve to compare the antagonist power of different modulators. In this case, the human neuroblastoma SH-SY5Y-TRPV1 cell line was treated with increasing concentrations of compound and the activity of the channel was determined by Ca²⁺-Fluorimetry. The obtained curve concentration-response served to calculate the IC50 value for each molecule. Molecules showing lower IC50 are more efficient modulating TRPV1 than those with higher IC50 values.

In one embodiment, optionally in combination with one or more features of the various embodiments described above or below, the known TRPV1 modulator is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts

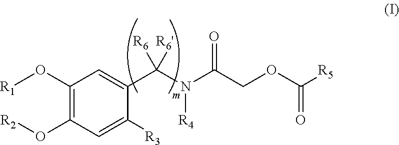

wherein
m is an integer selected from 1 to 3;
R1, R6 and R6 are independently selected from the group consisting of H, (C₁-C₃)alkyl, and unsaturated (C₂-C₈)hydrocarbon;
wherein (C₁-C₈)alkyl, and unsaturated (C₂-C₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHRs, —NHR6, —NHCOR6, —NHSO₂ R6, and —SO₂ NHR6;
R2 is H;
R3 is hydrogen or halogen;
R4 is selected from the group consisting of H, (C₁-C₈)alkyl and unsaturated (C₂-C₃)hydrocarbon;
wherein (C₁-C₈)alkyl and unsaturated (C₂-C₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHR6, —NHR6, —NHCOR6, —NHSO₂ R6, and —SO₂ NHR6;
R5 is selected from the group consisting of (C₃-C₂₈)alkyl, unsaturated (C₃-C₂₈)hydrocarbon, and

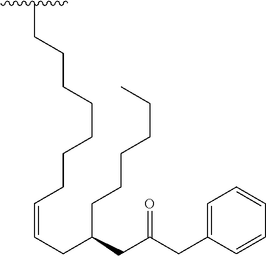 [SMILES: *CCCCCCC/C=C\C[C@@H](CCCCCC)CC(=O)Cc1ccccc1]

wherein (C₃-C₈)alkyl and unsaturated (C₃-C₂₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHR6, —NHR6, —NHCOR6, —NHSO₂ R6, and —SO₂ NHR6.

In one embodiment, optionally in combination with one or more features of the various embodiments described above or below, the known TRPV1 modulator is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts, wherein R1, R2, R3, R6, R6′ and m are as defined above, and R5 is selected from the group consisting of (C3-C28)alkyl, and unsaturated (C3-C28)hydrocarbon:
wherein (C₃-C₃)alkyl and unsaturated (C₃-C₂₈)hydrocarbon are optionally substituted with one or more substituents selected from the group consisting of halogen, —COOH, —OH, —NH₂, —COOR₆, —NO₂, —CF₃, —OCF₃, —CN, —OR₆, —CONH₂, —CONHR₆, —NHR₆, —NHCOR₆, —NHSO₂R₆, and —SO₂NHR₆;
or alternatively the compound of formula (I) is a compound selected from the group consisting of:
(Z)-2-((4-hydroxy-3-methoxy-benzyl)amino)-2-oxoethyl 12-(2-phenylacetoxy)octadec-9-enoate, and
(Z)-2-((4-hydroxy-2-iodo-5-methoxybenzyl)amino)-2-oxoethyl 12-(2-phenyl-acetoxy)octadec-9-enoate.

In one embodiment, optionally in combination with one or more features of the various embodiments described above or below, the known TRPV1 modulator is selected from the group consisting of a compound of formula (I) as defined above, which is selected from the group consisting of:
[1] 2-((4-hydroxy-3-methoxy-benzyl)amino)-2-oxoethyl butyrate;
[2] 2-((4-hydroxy-2-iodo-5-methoxybenzyl)amino)-2-oxoethyl butyrate;
[3] 2-((4-hydroxy-3-methoxy-benzyl)amino)-2-oxoethyl pentanoate;
[4] 2-((4-hydroxy-2-iodo-5-methoxybenzyl)amino)-2-oxoethyl pentanoate;
[5] 2-((4-hydroxy-3-methoxy-benzyl)amino)-2-oxoethyl 3-methylbutanoate;
[6] 2-((4-hydroxy-2-iodo-5-methoxybenzyl)amino)-2-oxoethyl 3-methyl-butanoate;
[7] 2-((4-hydroxy-3-methoxy-benzyl)amino)-2-oxoethyl hexanoate;
[8] 2-((4-hydroxy-2-iodo-5-methoxybenzyl)amino)-2-oxoethyl hexanoate;
[9] (2E,4E)-2-((4-hydroxy-3-methoxybenzyl)amino)-2-oxoethyl hexa-2,4-dienoate;
[10] (2E,4E)-2-((4-hydroxy-2-iodo-5-methoxybenzyl)amino)-2-oxoethyl hexa-2,4-dienoate;
[11] 2-((4-hydroxy-3-methoxy-benzyl)amino)-2-oxoethyl heptanoate;
[12] 2-((4-hydroxy-2-iodo-5-methoxybenzyl)amino)-2-oxoethyl heptanoate;
[13] 2-((4-hydroxy-3-methoxy-benzyl)amino)-2-oxoethyl octanoate;
[14] 2-((4-hydroxy-2-iodo-5-methoxybenzyl)amino)-2-oxoethyl octanoate;
[15] 2-((4-hydroxy-3-methoxybenzyl)amino)-2-oxoethyl nonanoate;
[16] 2-((4-hydroxy-2-iodo-5-methoxybenzyl)amino)-2-oxoethyl nonanoate;
[17] (E)-2-((4-hydroxy-3-methoxy-benzyl)amino)-2-oxoethyl 3,7-dimethylocta-2,6-dienoate;
[18] (E)-2-((4-hydroxy-2-iodo-5-methoxybenzyl)amino)-2-oxoethyl 3,7-dimethylocta-2,6-dienoate;
[19] 2-((4-hydroxy-3-methoxy-benzyl)amino)-2-oxoethyl decanoate;
[20] 2-((4-hydroxy-2-iodo-5-methoxybenzyl)amino)-2-oxoethyl decanoate;
[21] 2-((4-hydroxy-3-methoxy-benzyl)amino)-2-oxoethyl dodecanoate;
[22] 2-((4-hydroxy-2-iodo-5-methoxybenzyl)amino)-2-oxoethyl dodecanoate;
[23] 2-((4-hydroxy-3-methoxy-benzyl)amino)-2-oxoethyl pentadecanoate;
[24] 2-((4-hydroxy-2-iodo-5-methoxybenzyl)amino)-2-oxoethyl pentadecanoate;
[25] 2-((4-hydroxy-3-methoxy-benzyl)amino)-2-oxoethyl stearate;
[26] 2-((4-hydroxy-2-iodo-5-methoxybenzyl)amino)-2-oxoethyl stearate;
[27] 2-((4-hydroxy-3-methoxy-benzyl)amino)-2-oxoethyl oleate;
[28] 2-((4-hydroxy-2-iodo-5-methoxybenzyl)amino)-2-oxoethyl oleate;
[29] (R,Z)-2-((4-hydroxy-3-methoxybenzyl)amino)-2-oxoethyl 12-hydroxyoctadec-9-enoate;
[30] (R,Z)-2-((4-hydroxy-2-iodo-5-methoxybenzyl)amino)-2-oxoethyl 12-hydroxyoctadec-9-enoate;
[31] (Z)-2-((4-hydroxy-3-methoxy-benzyl)amino)-2-oxoethyl 12-(2-phenylacetoxy)octadec-9-enoate;
[32] (Z)-2-((4-hydroxy-2-iodo-5-methoxybenzyl)amino)-2-oxoethyl 12-(2-phenyl-acetoxy)octadec-9-enoate;
[33] (Z)-2-((4-hydroxy-3-methoxybenzyl)amino)-2-oxoethyl docos-13-enoate;
[34] (Z)-2-((4-hydroxy-2-iodo-5-methoxybenzyl)amino)-2-oxoethyl docos-13-enoate;
[35] (5Z,8Z,11Z,14Z)-2-((4-hydroxy-3-methoxy-benzyl)amino)-2-oxoethyl icosa-5,8,11,14-tetraenoate;
[36] (5Z,8Z,11Z,14Z)-2-((4-hydroxy-2-iodo-5-methoxy-benzyl)amino)-2-oxoethyl icosa-5,8,11,14-tetraenoate;
[37] (4E,8E,12E,16E)-2-((4-hydroxy-3-methoxybenzyl)-amino)-2-oxoethyl 4,8,13,17,21-pentamethyldocosa-4,8,12,16,20-pentaenoate;
[38] (4E,8E,12E,16E)-2-((4-hydroxy-2-iodo-5-methoxy-benzyl)amino)-2-oxoethyl 4,8,13,17,21-pentamethyl-docosa-4,8,12,16,20-pentaenoate;
[39] (E)-2-((3,4-dihydroxybenzyl)-amino)-2-oxoethyl 3,7-dimethylocta-2,6-dienoate;
[40] (E)-2-((4,5-dihydroxy-2-iodo-benzyl)amino)-2-oxoethyl 3,7-dimethylocta-2,6-dienoate;
[41] (4E,8E,12E,16E)-2-((3,4-dihydroxybenzyl)amino)-2-oxoethyl 4,8,13,17,21-pentamethyldocosa-4,8,12,16,20-pentaenoate;
[42] (4E,8E,12E,16E)-2-((4,5-dihydroxy-2-iodobenzyl)-amino)-2-oxoethyl 4,8,13,17,21-pentamethyl-docosa-4,8,12,16,20-pentaenoate;
[43] (E)-2-((3,4-dihydroxy-phenethyl)amino)-2-oxoethyl 3,7-dimethylocta-2,6-dienoate;
[44] (E)-2-((4,5-dihydroxy-2-iodophenethyl)amino)-2-oxoethyl 3,7-dimethylocta-2,6-dienoate;
[45] (4E,8E,12E,16E)-2-((3,4-dihydroxyphenethyl)amino)-2-oxoethyl 4,8,13,17,21-pentamethyldocosa-4,8,12,16,20-pentaenoate;
[46] (4E,8E,12E,16E)-2-((4,5-dihydroxy-2-iodophenethyl)-amino)-2-oxoethyl 4,8,13,17,21-pentamethyl-docosa-4,8,12,16,20-pentaenoate;
[47] 2-((3,4-dihydroxyphenethyl)-amino)-2-oxoethyl oleate;
[48] 2-((4,5-dihydroxy-2-iodophenethyl)amino)-2-oxoethyl oleate;
[49] (5Z,8Z,11Z,14Z)-2-((3,4-dihydroxyphenethyl)amino)-2-oxoethyl icosa-5,8,11,14-tetraenoate; and
[50] (5Z,8Z,11Z,14Z)-2-((4,5-dihydroxy-2-iodophenethyl)-amino)-2-oxoethyl icosa-5,8,11,14-tetraenoate.

In one embodiment, optionally in combination with one or more features of the various embodiments described above or below, the known TRPV1 modulator is 2-((4-hydroxy-2-iodo-5-methoxybenzyl)amino)-2-oxoethyl nonanoate (Calmapsin®).

The compounds of formula (I) as defined above may be prepared by a process which comprises:
a) reacting a compound of formula (II) with a compound of formula (III)

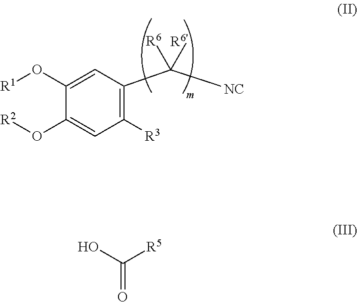

wherein R1, R2, R3, R5, R6, R6′ and m are as defined above; in the presence of formaldehyde to yield a compound of formula (I);
b) optionally converting, in one or a plurality of steps, the compound of formula (I) thus obtained into another compound of formula (I); and
c) optionally reacting the compound of formula (I) obtained in any of the steps a) or b) with a base or with an acid to give the corresponding salt.

Some of the compounds of formula (I) wherein R3 is H, are TRPV1 agonists. Some of the compounds of the invention, in particular the compounds of formula (I) wherein R3 is halogen, particularly iodine, are TRPV1 antagonists.

For the purposes of the invention, TRPV1 agonists as defined herein are capable of activating TRPV1 with an EC50 value≤10 μM, preferably an EC50 value≤8, more preferably ≤1 μM, more preferably ≤500 nM, and TRPV1 antagonists as defined herein are capable of inhibiting TRPV1 with an IC50 value s 100, preferably an IC50 value≤50 μM, preferably ≤30 μM, more preferably ≤10 μM, where the activation/inhibition of TRPV1 is measured by Calcium microfluorometry assays (e.g. in vitro assays).

Thus, the known TRPV1 modulators, via direct desensitization of the receptor in the case of TRPV1 agonists, or by blocking TRPV1 in the case of antagonists, cause a loss of TRPV1 activity. The use of TRPV1 antagonists has the further advantage that they do not cause a burning sensation when applied.

In accordance with one embodiment, optionally in combination with one or more features of the various embodiments described above or below, the composition comprises a synergistic combination of a known neuronal exocytosis inhibitor; and a known TRPV1 modulator which is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts, as defined above.

In one particular embodiment, the composition comprises a synergistic combination of a known neuronal exocytosis inhibitor, and a known TRPV1 modulator which is selected from the group consisting of a compound from [1] to [50] as above, or a pharmaceutically or cosmetically acceptable salt thereof, or any stereoisomer or mixtures thereof, either of the compound of formula (I) or of any of its pharmaceutically or cosmetically acceptable salts.

In one particular embodiment, the composition comprises a synergistic combination of a known neuronal exocytosis inhibitor, and a known TRPV1 modulator which is 2-((4-hydroxy-2-iodo-5-methoxybenzyl)amino)-2-oxoethyl nonanoate.

In one embodiment, optionally in combination with one or more features of the various embodiments described above or below, the known neuronal exocytosis inhibitor is able to at least partially inhibit neuronal exocytosis; which in a particular embodiment it takes through a mechanism that involves interfering with the assembly of the fusion protein complex (SNARE) and/or its thermal destabilization.

In one embodiment, optionally in combination with one or more features of the various embodiments described above or below, the known neuronal exocytosis inhibitor is a known peptide derived from SNAP-25 protein, sintaxin protein, synaptobrevin protein (VAMP), or Munc protein.

Examples of known neuronal exocytosis inhibitors derived from Munc protein are described e.g. in WO2019166347 related to peptides capable of interfering in the Munc18-Syntaxin-1 complex interaction.

In one embodiment, optionally in combination with one or more features of the various embodiments described above or below, the known neuronal exocitosis inhibitor is selected from the group consisting of known peptides consisting of an amino acid sequence deriving from the N-terminal domain of protein SNAP-25, known peptides consisting of an amino acid sequence deriving from the C-terminal domain of protein SNAP-25, and mixtures thereof.

In a particular embodiment, optionally in combination with one or more features of the various embodiments described above or below, the known neuronal exocitosis inhibitor is selected from the group consisting of
i. a peptide consisting of an amino acid sequence selected from the amino acid sequence of SEQ. ID NO. 1 or the amino acid sequence of SEQ. ID NO. 2;
ii. the peptides defined by SEQ. ID NO. 1 or SEQ. ID NO. 2 wherein the amino acid at the N-terminus of the peptide is acetylated, and, optionally, wherein the amino acid at the C-terminus of the peptide is aminated;
iii. the peptides defined by SEQ. ID NO. 1 or SEQ. ID NO. 2 wherein the amino acid at the C-terminus of the peptide is aminated, and, optionally, wherein the amino acid at the C-terminus of the peptide is acetylated;
iv. the peptides defined by SEQ. ID NO. 1 or SEQ. ID NO. 2 wherein further contains a reversible chemical modification that increases its bioavailability and facilitates its permeation through the blood brain barrier and epithelial tissue;
v. a substantially homologous peptide to the peptide defined by SEQ. ID NO. 1 or SEQ. ID NO. 2;
vi. a pharmaceutically or cosmetically acceptable salt of the peptide defined by SEQ. ID NO. 1 or SEQ. ID NO. 2;
vii. a peptide defined by SEQ. ID NO. 1 or SEQ. ID NO. 2 that has undergone reversible chemical modifications.

In one embodiment, optionally in combination with one or more features of the various embodiments described above or below, the known neuronal exocitosis inhibitor is selected from the group consisting of a peptide consisting of an amino acid sequence deriving from the N-terminal domain of protein SNAP-25 having a sequence of 3 to 30 adjacent amino acids contained in SNAP-25 protein, including peptides which are substantially homologous to the peptides of SEQ. ID NO. 1 or SEQ. ID NO. 2.

In the sense used in this description, the expression “substantially homologous” means that the peptide in question has a homology level, as far as amino acids are concerned, of at least 60%, and preferably of at least 80%, and even more preferably, of at least 95%.

In one embodiment, optionally in combination with one or more features of the various embodiments described above or below, the known neuronal exocitosis inhibitor is selected from the group consisting of a peptide consisting of an amino acid sequence deriving from the N-terminal domain of protein SNAP-25 including peptides which are functionally equivalent to the peptides of SEQ. ID NO. 1 or SEQ. ID NO. 2.

In the sense used in this description, the expression “functionally equivalent” means that the peptide in question has at least one of the biological activities of the peptide of SEQ. ID NO. 1 or SEQ. ID NO. 2, such as, for example, the ability to at least partially inhibit neuronal exocytosis.

The amino acids that make up the structural units of the peptide may have D- or L-configuration. The amino acid from the amino end may have an acetylated terminal amino group, and the amino acid from the carboxyl end may have an amidated terminal carboxyl group. Therefore, this invention also includes derivatives of a known neuronal exocytosis inhibitor which may be selected from the group consisting of a peptide consisting of an amino acid sequence deriving from the N-terminal domain of protein SNAP-25 in which the amino-terminal end is acetylated and/or in those where the carboxy-terminal end is amidated.

Within the scope of this invention are cosmetically and/or pharmaceutically acceptable salts of the known neuronal exocytosis inhibitor, which in one embodiment is selected from the group consisting of a peptide consisting of an amino acid sequence deriving from the N-terminal domain of protein SNAP-25.

In addition, the known neuronal exocytosis inhibitor, which in one embodiment is selected from the group consisting of a peptide consisting of an amino acid sequence deriving from the N-terminal domain of protein SNAP-25 may undergo reversible chemical modifications in order to increase its bioavailability (including stability and fat solubility) and its ease in passing through the blood-brain barrier and epithelial tissue.

In one embodiment, optionally in combination with one or more features of the various embodiments described above or below, the combination of the invention comprises or consists a known neuronal exocytosis inhibitor selected from
i. a peptide consisting of an amino acid sequence selected from the amino acid sequence of SEQ. ID NO. 1 or the amino acid sequence of SEQ. ID NO. 2;
ii. the peptides defined by SEQ. ID NO. 1 or SEQ. ID NO. 2 wherein the amino acid at the N-terminus of the peptide is acetylated, and, optionally, wherein the amino acid at the C-terminus of the peptide is aminated;
iii. the peptides defined by SEQ. ID NO. 1 or SEQ. ID NO. 2 wherein the amino acid at the C-terminus of the peptide is aminated, and, optionally, wherein the amino acid at the C-terminus of the peptide is acetylated;
iv. the peptides defined by SEQ. ID NO. 1 or SEQ. ID NO. 2 wherein further contains a reversible chemical modification that increases its bioavailability and facilitates its permeation through the blood brain barrier and epithelial tissue;
v. —a substantially homologous peptide to the peptide defined by SEQ. ID NO. 1 or SEQ. ID NO. 2;
vi. —a pharmaceutically or cosmetically acceptable salt of the peptide defined by SEQ. ID NO. 1 or SEQ. ID NO. 2;
vii. —a peptide defined by SEQ. ID NO. 1 or SEQ. ID NO. 2 that has undergone reversible chemical modifications;
   and
   a known TRPV1 modulator selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts as defined above.

In one embodiment, optionally in combination with one or more features of the various embodiments described above or below, the composition according to the invention comprises the combination of
a) a known neuronal exocytosis inhibitor selected from
   i) a peptide consisting of an amino acid sequence selected from the amino acid sequence of SEQ. ID NO. 1 or the amino acid sequence of SEQ. ID NO. 2;
   ii) the peptides defined by SEQ. ID NO. 1 or SEQ. ID NO. 2 wherein the amino acid at the N-terminus of the peptide is acetylated, and, optionally, wherein the amino acid at the C-terminus of the peptide is aminated; and
   iii) the peptides defined by SEQ. ID NO. 1 or SEQ. ID NO. 2 wherein the amino acid at the C-terminus of the peptide is aminated, and, optionally, wherein the amino acid at the C-terminus of the peptide is acetylated;
      and
b) the known TRPV1 modulator 2-((4-hydroxy-2-iodo-5-methoxybenzyl)amino)-2-oxoethyl nonanoate.

In one particular embodiment, the known neuronal exocytosis inhibitors is the acetylated form of the peptide of SEQ. ID. NO 1, commercialized under the tradename Argireline® by Lipotec, S.A. (Ac-Glu-Glu-Met-Gln-Arg-Arg); and the known TRPV1 modulator 2-((4-hydroxy-2-iodo-5-methoxybenzyl)amino)-2-oxoethyl nonanoate.

In one embodiment, optionally in combination with one or more features of the various embodiments described above or below, the weight ratio between the known neuronal exocytosis and the known TRPV1 modulator is from 10:1 to 1:1, preferably from 7.5:1 to 2.5:1, being particularly preferably 5:1.

In one embodiment, optionally in combination with one or more features of the various embodiments described above or below, the combination of the invention comprises or consists of a known neuronal exocytosis inhibitor in a concentration between 0.5% and 0.005% in weight, preferably between 0.1 and 0.01% in weight with regards to the total weight of the composition; and a known TRPV1 modulator in a concentration between 0.1-0.001% in weight, preferably between 0.02 and 0.002% in weight with regards to the total weight of the composition.

The neuronal-exocytosis (neurosecretion) inhibiting capabilities of the known neuronal exocytosis inhibitors became evident through a test that evaluates the strength of said compounds in inhibiting the release of catecholamines induced by calcium in chromaffin cells permeabilized with the detergent digitonin, in accordance with the method described by Gutiérrez et al. (FEBS Lett 1995, 372, 39-43, and J. Biol. Chem. 1997, 272, 2634-2639); as well as an in vitro reconstitution test of the fusion complex (SNARE).

The neuronal-exocytosis (neurosecretion) inhibiting capabilities of the known neuronal exocytosis inhibitors may also be known evaluating the inhibition of α-calcitonin gene-related peptide (α-CGRP) exocytotic release from intact primary sensory neurons in accordance with the method described by Butrón et al. (Bioorg Chem. 2021; 115:105231. doi:10.1016/j.bioorg.2021.105231. Epub 2021 Jul. 31).

Briefly, the chromaffin cell cultures are incubated with [3 H]-epinephrine and [3 H]-norepinephrine, are permeabilized with 20 μM digitonin, and stimulated with calcium (10 μM), in the presence of the compounds to be tested, and the amount of radioactivity released by the cells to the extracellular medium, which is a reflection of the exocytosis of [3 H]-epinephrine and [3 H]-norepinephrine, is measured.

The results obtained in inhibiting the release of catecholamines in permeabilized chromaffin cells for the peptides of SEQ. ID NO. 1 and SEQ. ID NO. 2 were the following:
a) the peptide in SEQ. ID NO. 1, from the amino end of SNAP-25, at a concentration of 1 mM, blocked approximately 20% of the release of catecholamines in permeabilized chromaffin cells;
b) the peptide in SEQ. ID NO. 2, from the amino end of SNAP-25, at a concentration of 1 mM, inhibited approximately 35% of the release of catecholamines in the permeabilized chromaffin cells.

These results indicate that both peptides inhibit catecholamine exocytosis.

An additional test of in vitro reconstitution was performed to determine the ability of the peptides to interfere with the formation and stability of the fusion complex (SNARE). The test consists of evaluating the in vitro reconstitution of the fusion protein complex with recombinant proteins produced in Escherichia coli. The reconstitution tests, based on ELISA (Enzyme-Linked Immuno Assay) methods, involve the immobilization of protein SNAP-25 in 96-well plates and the subsequent formation of the fusion protein complex by adding the proteins VAMP and syntaxin in the presence and/or absence of the peptides of the invention. The detection of the complex was performed using an antibody against protein syntaxin (anti-syntaxin), followed by an antibody which recognizes the anti-syntaxin antibody, covalently tagged with a peroxidase. The amount of fusion protein complex was tracked by adding 1,2-phenylenediamine dichloride, whose reaction with the peroxidase produces a product with an orangish-yellow color that absorbs 492 nm in an acid medium.

The data obtained for peptides of SEQ. ID NO. 1 and SEQ. ID NO. 2 show that the presence of the peptides during the assembly of the fusion complex causes a significant decrease in same. Therefore, the mechanism of the action of said peptides seems to imply that said peptides interfere with the formation and/or stability of the fusion protein complex (SNARE).

In particular, the peptide of SEQ. ID NO. 1, at a concentration of 1 mM, blocked approximately 20% of the release of catecholamines (epinephrine and norepinephrine) in permeabilized chromaffin cells, while the peptide of SEQ. ID NO. 2, at a concentration of 1 mM, inhibited approximately 35% of the release of catecholamines in the permeabilized chromaffin cells.

The composition according to the invention may be administered by any means which causes contact between the compounds and the site of action in a mammal's body, preferably that of a human being, and in the form of a composition which contains them.

To this regard, in accordance with an embodiment of the present invention, the invention relates to a cosmetic or pharmaceutical composition which comprises an effective amount of the composition as defined herein together with at least one cosmetically or pharmaceutically acceptable excipients or carriers. These compositions can be prepared by conventional means known to persons skilled in the art.

The particular dose of composition administered according to this invention will of course be determined by the particular circumstances surrounding the case, including the compounds administered, the route of administration, the particular condition being treated, age, state of the patient, and the similar considerations. Accordingly, a therapeutically effective amount of a compound may be an amount which is sufficient to treat a disease or disorder, delay the onset or progression of a disease or disorder, and/or alleviate one or more symptoms of the disease or disorder, when administered to a subject suffering from said disease or disorder. The precise effective amount for a subject will depend upon a variety of factors such as the subject's body weight, size and health, the nature and extent of the condition to be treated, and the therapeutic or combination of therapeutics selected for administration. Therapeutically effective amounts for a given situation can be determined by routine experimentation that is within the skill and judgement of the clinician.

The election of the pharmaceutical formulation will depend upon the nature of the active compound and its route of administration. Any route of administration may be used, for example topical, oral and parenteral administration.

Parenteral administration includes intravenous, intra-arterial, subcutaneous, intraperitoneal or intramuscular. Parenteral administration can be in the form of a single bolus dose, or may be, for example, by a continuous perfusion pump.

The compositions can be formulated as to provide quick (immediate), sustained or delayed release of the active ingredient after administration to the patient by using methods known in the art.

Examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, and methyl cellulose. The pharmaceutical compositions can additionally include further pharmaceutically acceptable excipients including: lubricating agents such as talc, magnesium stearate and mineral oil; wetting agents; emulsifying and suspending agents; preserving agents such as methyl- and propylhydroxybenzoates; sweetening agents; flavouring agents; and colouring agents.

Pharmaceutical compositions suitable for parenteral administration include sterile aqueous solutions or suspensions, or can be alternatively prepared in lyophilized form for extemporaneous preparation of a solution or suspension using a sterile aqueous carrier prior to use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

The pharmaceutical compositions, like oral and parenteral compositions, can be formulated in unit dosage forms for ease of administration and uniformity of dosage. As used herein, “unit dosage forms” refers to physically discrete units suitable as unitary dosages for administration to subjects, each unit containing a predetermined quantity of active ingredient calculated to produce the desired therapeutic effect, in association with one or more suitable pharmaceutical carriers.

In therapeutic applications, pharmaceutical compositions are to be administered in a manner appropriate to the disease to be treated, as determined by a person skilled in the medical arts. An appropriate dose and suitable duration and frequency of administration will be determined by such factors as the condition of the patient, the type and severity of the disease, the particular form of the active ingredient and the method of administration, among others. In general, an appropriate dose and administration regimen provides the pharmaceutical composition in an amount sufficient to provide therapeutic benefit, for example an improved clinical outcome, such as more frequent complete or partial remissions, or longer disease-free and/or overall survival, or lessening of symptoms severity, or any other objectively identifiable improvement as noted by the clinician.

The pharmaceutical compositions of the invention can be included in a container, pack or dispenser together with instructions for administration.

The combination of the present invention can be in form of a topical pharmaceutical or cosmetic composition. The cosmetic or pharmaceutical compositions which contain the combination of the invention, can be used in different types of compositions of topical application which optionally include cosmetically or pharmaceutically acceptable excipients necessary for formulating the desired administration form. A person skilled in the art knows the different excipients which can be used in the cosmetic or pharmaceutical compositions which contain the compounds of the invention.

The compositions of topical application can be produced in any solid, liquid or semi-solid formulation, such as and not restricted to, creams, multiple emulsions such as and not restricted to, oil and/or silicone in water emulsions, water-in-oil and/or silicone emulsions, water/oil/water or water/silicone/water type emulsions, and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols (sprays), including leave-on and rinse-off formulations. These topical application formulations can be incorporated using techniques known by the person skilled in the art into different types of solid accessories such as and not restricted to, bandages, gauzes, t-shirts, socks, tights, underwear, girdles, gloves, diapers, sanitary napkins, dressings, bedspreads, wipes, adhesive patches, non-adhesive patches, occlusive patches, microelectric patches or face masks, or they can be incorporated into different make-up products such as make-up foundation, such as fluid foundations and compact foundations, make-up removal lotions, make-up removal milks, under-eye concealers, eye shadows, lipsticks, lip protectors, lip gloss and powders, among others.

In a particular embodiment, the topical composition is a pharmaceutical composition comprising an effective amount of the combination as defined above together with one or more topical pharmaceutically acceptable excipients or carriers.

In another particular embodiment, the topical composition is a cosmetic composition comprising an effective amount of the combination as defined above together with one or more topical cosmetically acceptable excipients or carriers.

The topical compositions defined above comprise appropriate excipients or carriers for topical administration that can be pharmaceutical or cosmetic excipients, including, but not limited to, repairing cutaneous barrier function agent, a hydrating agent, an emollient, an emulsifier, a thickener, a humectant, a pH-regulating agent, an antioxidant, a preservative agent, a vehicle, or their mixtures. The excipients or carriers used have affinity for the skin, are well tolerated, stable, and are used in an amount adequate to provide the desired consistency, and ease application. The topical compositions of the invention can be formulated in several forms that include, but are not limited to, solutions, aerosols and non-aerosol sprays, shaving creams, powders, mousses, lotions, gels, sticks, ointments, pastes, creams, shampoos, shower gel, body washes or face washes.

Topical compositions of the present invention can be prepared according to methods well known in the state of the art. The appropriate excipients and/or carriers, and their amounts, can readily be determined by those skilled in the art according to the type of formulation being prepared.

The cosmetic or pharmaceutical compositions according to the present invention may also contain a cosmetically or pharmaceutically effective amount of at least one cosmetic and/or absorbent and/or body odor masking deodorant and/or antiperspirant agent, perfume and/or perfumed oil.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

### EXAMPLES

### Example 1. Combination of Calmapsin® and Argireline® on Excessive Sweating or Perspiration

To carry out the experimental part, a cream containing both active ingredients and several control creams (1: without Calmpasin®, 2: without Argeriline and 3: without Calmapsin® and Argireline) were developed. The composition of the cream is shown below in Table 1.

TABLE 1
Composition of the products used in the study.

| | Cuantitative Composition |
|---|---|
| Cream 1 | 78.64 g Cyclopentasiloxane and dimethicone crosspolymer |
| (Calmapsin + | 9.40 g Carpylyl dimethicone |
| Argireline) | 9.40 g Dimethicone 5 sct |
| | 1.00 g PEG-10 Dimethicone |
| | 1.00 g Diethylene glicol monoethyl ether |
| | 1.00 g Transcutol |
| | 0.50 g Water |
| | 0.05 g Argireline |
| | 0.01 g Hydroxymethoxyiodobenzy glycolamide pelargonate |
| | Total 100 g |
| Cream 2 | 78.65 g Cyclopentasiloxane and dimethicone crosspolymer |
| (Without | 9.40 g Carpylyl dimethicone |
| Calmapsin) | 9.40 g Dimethicone 5 sct |
| | 1.00 g PEG-10 Dimethicone |
| | 1.00 g Diethylene glicol monoethyl ether |
| | 1.00 g Transcutol |
| | 0.50 g Water |
| | 0.05 g Argireline |
| | Total 100 g |
| Cream 3 | 78.69 g Cyclopentasiloxane and dimethicone crosspolymer |
| (Without | 9.40 g Carpylyl dimethicone |
| Argireline) | 9.40 g Dimethicone 5 sct |
| | 1.00 g PEG-10 Dimethicone |
| | 1.00 g Diethylene glicol monoethyl ether |
| | 1.00 g Transcutol |
| | 0.50 g Water |
| | 0.01 g Hydroxymethoxyiodobenzy glycolamide pelargonate |
| | Total 100 g |
| Cream 4 | 78.70 g Cyclopentasiloxane and dimethicone crosspolymer |
| (Without | 9.40 g Carpylyl dimethicone |
| Calmapsin ® nor | 9.40 g Dimethicone 5 sct |
| Argireline) | 1.00 g PEG-10 Dimethicone |
| | 1.00 g Diethylene glicol monoethyl ether |
| | 1.00 g Transcutol |
| | 0.50 g Water |
| | Total 100 g |

The cream was tested on a group of volunteers who reported excessive sweating and sensitive skin. The application pattern was twice daily for 4 weeks (28 days). Before starting the study, trans-epidermal water loss was quantified using a TEWELmeter (DO). After 4 weeks, the procedure was repeated (D28). The results and the statistical significance of the differences found are shown in Table 2.

TABLE 2
measurement of trans-epidermal water loss by TEWELmeter in subjects participating
in the study before starting (D 0) and after 4 weeks using the product (D 29).

| | | | | | Std. | | | P- |
|---|---|---|---|---|---|---|---|---|
| Cream | | N | Mean | Median | deviation | Minimun | Maximum | Value* |
| 1 | D 0 | 24 | 45.69 | 48.15 | 10.32 | 20.05 | 60.10 | <0.001 |
| | D 29 | 24 | 29.59 | 29.99 | 15.51 | 7.45 | 57.20 | |
| | D 29- | 24 | −16.11 | −13.80 | 18.13 | −50.00 | 25.23 | |
| | D 0 | | | | | | | |
| 2 | D 0 | 5 | 42.40 | 44.18 | 8.61 | 31.97 | 53.10 | 0.301 |
| | D 29 | 5 | 33.41 | 30.44 | 14.25 | 15.23 | 57.20 | |
| | D 29- | 5 | −8.99 | −11.95 | 19.10 | −30.57 | 25.23 | |
| | D 0 | | | | | | | |
| 3 | D 0 | 5 | 44.36 | 43.27 | 9.22 | 28.89 | 56.15 | 0.461 |
| | D 29 | 5 | 38.31 | 35.74 | 14.82 | 12.94 | 52.67 | |
| | D 29- | 5 | −6.05 | −7.53 | 19.36 | −30.50 | 13.65 | |
| | D 0 | | | | | | | |
| 4 | D 0 | 5 | 54.69 | 52.98 | 8.65 | 39.49 | 61.08 | 0.523 |
| | D 29 | 5 | 42.03 | 36.84 | 12.26 | 27.90 | 56.15 | |
| | D 29- | 5 | −12.66 | −16.44 | 14.60 | −29.42 | 18.39 | |
| | D 0 | | | | | | | |

*P-Value < 0.05: significant difference

As it can be observed in Table 2, there is a significant decrease in the trans-epidermal water loss after four weeks using cream 1, meaning that people using cream 1 experienced a significant decrease in perspiration. None of the other three creams (cream 2-4) achieved a significant decrease of water loss.

None of the participants in the study reported any uncomfortable or uncomfortable sensations after using the product and none developed adverse skin reactions and it was classified as “very good skin compatibility”.

### Subjective Measurement of the Product's Effects on Perspiration

Study participants completed a questionnaire on the properties of the product and its efficacy after 4 weeks of use. 83% of the participants ( 20/24) who used Cream 1 (Calmapsin®+Argireline®) stated that the product reduced their excessive sweating and regulated their perspiration and 79% ( 19/24) of them stated that they noticed an improvement in the odor of their sweat after using the product. Sixty percent (⅗) of the participants who used Cream 2 (without Calpasin®) reported that the cream reduced their excessive sweating and regulated their perspiration. Finally, none of the volunteers using Creams 3 (without Argireline) and 4 (without Calmapsin® or Argireline®) felt a reduction in sweating or perspiration after using the product.

### Measurement of Sweating Using the Minor's Test

In addition, a Minor test was carried out. This test consists of applying an iodine solution to the area of excessive sweating. After a few minutes, starch powder is applied. When sweating begins, the iodine changes color, leaving a trace that allows identification of the areas with increased sweating.

The volunteers underwent the Minor's Test after 4 weeks using Cream 1 (Calmapsin®+Argireline®) only on one armpit. Thus, the other armpit served as a control. There were observed a significant difference in the amount of sweating between the area treated with the product (left arm) and the control area (right arm).

### REFERENCES CITED IN THE APPLICATION

EP1180524
EP1856139
EP3621950
WO2019166347
FEBS Lett 1995, 372, 39-43
J. Biol. Chem. 1997, 272, 2634-2639
Bioorg Chem. 2021; 115:105231

For reasons of completeness, various aspects of the invention are set out in the following numbered clauses:

Clause 1. A composition comprising a synergistic combination of:
a) a known neuronal exocytosis inhibitor; and
b) a known TRPV1 modulator.

Clause 2. The composition according to clause 1 wherein the known TRPV1 modulator is selected from the group of known TRPV1 agonists capable of desensitizing TRPV1 with an EC50 value≤10 μM, and TRPV1 antagonists capable of inhibiting TRPV1 with an IC50 value≤100 μM; where the activation/inhibition of TRPV1 is measured by calcium microfluorometry assays as described in the description.

Clause 3. The composition according to any of clauses 1 to 2, wherein the known neuronal-exocytosis inhibitor is selected from those which at a concentration of 1 mM inhibit from 15% to 45% of the release of catecholamines in the permeabilized chromaffin cells; where the inhibition of neural-exocytosis is measured by evaluating the strength of said compounds in inhibiting the release of catecholamines induced by calcium in chromaffin cells permeabilized with the detergent digitonin in accordance with the method described in the description.

Clause 4. The composition according to any of clauses 1 to 3, wherein the known TRPV1 modulator is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts

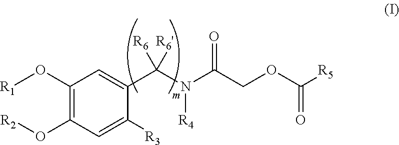

wherein
m is an integer selected from 1 to 3;
R1, R6 and R6 are independently selected from the group consisting of H, (C₁-C₃)alkyl, and unsaturated (C₂-C₈)hydrocarbon;
wherein (C₁-C₈)alkyl, and unsaturated (C₂-C₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHR6, —NHR6, —NHCOR6, —NHSO₂R6, and —SO₂NHR6;
R2 is H;
R3 is hydrogen or halogen;
R4 is selected from the group consisting of H, (C₁-C₈)alkyl and unsaturated (C₂-C₃)hydrocarbon;
wherein (C₁-C₈)alkyl and unsaturated (C₂-C₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHR6, —NHR6, —NHCOR6, —NHSO₂R6, and —SO₂NHR6;
R5 is selected from the group consisting of (C₃-C₂₈)alkyl, unsaturated (C₃-C₂₈)hydrocarbon, and

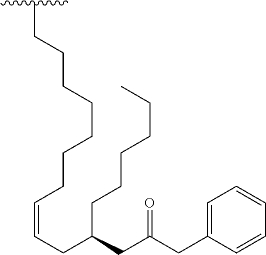 [SMILES: *CCCCCCC/C=C\C[C@@H](CCCCCC)CC(=O)Cc1ccccc1]

wherein (C₃-C₈)alkyl and unsaturated (C₃-C₂₈)hydrocarbon are optionally substituted with one or more substituents selected from the group consisting of halogen, —COOH, —OH, —NH₂, —COOR6, —NO₂, —CF₃, —OCF₃, —CN, —OR6, —CONH₂, —CONHR6, —NHR6, —NHCOR6, —NHSO₂R6, and —SO₂NHR6.

Clause 5. The composition according to any of clauses 1-4, wherein the known TRPV1 modulator is selected from the group consisting of a compound of formula (I) according to claim 2, which is selected from the group consisting of:
2-((4-hydroxy-3-methoxy-benzyl)amino)-2-oxoethyl butyrate;
2-((4-hydroxy-2-iodo-5-methoxybenzyl)amino)-2-oxoethyl butyrate;
2-((4-hydroxy-3-methoxy-benzyl)amino)-2-oxoethyl pentanoate;
2-((4-hydroxy-2-iodo-5-methoxybenzyl)amino)-2-oxoethyl pentanoate;
2-((4-hydroxy-3-methoxy-benzyl)amino)-2-oxoethyl 3-methylbutanoate;
2-((4-hydroxy-2-iodo-5-methoxybenzyl)amino)-2-oxoethyl 3-methyl-butanoate;
2-((4-hydroxy-3-methoxy-benzyl)amino)-2-oxoethyl hexanoate;
2-((4-hydroxy-2-iodo-5-methoxybenzyl)amino)-2-oxoethyl hexanoate;
(2E,4E)-2-((4-hydroxy-3-methoxybenzyl)amino)-2-oxoethyl hexa-2,4-dienoate;
(2E,4E)-2-((4-hydroxy-2-iodo-5-methoxybenzyl)amino)-2-oxoethyl hexa-2,4-dienoate;
2-((4-hydroxy-3-methoxy-benzyl)amino)-2-oxoethyl heptanoate;
2-((4-hydroxy-2-iodo-5-methoxybenzyl)amino)-2-oxoethyl heptanoate;
2-((4-hydroxy-3-methoxy-benzyl)amino)-2-oxoethyl octanoate;
2-((4-hydroxy-2-iodo-5-methoxybenzyl)amino)-2-oxoethyl octanoate;
2-((4-hydroxy-3-methoxybenzyl)amino)-2-oxoethyl nonanoate;
2-((4-hydroxy-2-iodo-5-methoxybenzyl)amino)-2-oxoethyl nonanoate;
(E)-2-((4-hydroxy-3-methoxy-benzyl)amino)-2-oxoethyl 3,7-dimethylocta-2,6-dienoate;
(E)-2-((4-hydroxy-2-iodo-5-methoxybenzyl)amino)-2-oxoethyl 3,7-dimethylocta-2,6-dienoate;
2-((4-hydroxy-3-methoxy-benzyl)amino)-2-oxoethyl decanoate;
2-((4-hydroxy-2-iodo-5-methoxybenzyl)amino)-2-oxoethyl decanoate;
2-((4-hydroxy-3-methoxy-benzyl)amino)-2-oxoethyl dodecanoate;
2-((4-hydroxy-2-iodo-5-methoxybenzyl)amino)-2-oxoethyl dodecanoate;
2-((4-hydroxy-3-methoxy-benzyl)amino)-2-oxoethyl pentadecanoate;
2-((4-hydroxy-2-iodo-5-methoxybenzyl)amino)-2-oxoethyl pentadecanoate;
2-((4-hydroxy-3-methoxy-benzyl)amino)-2-oxoethyl stearate;
2-((4-hydroxy-2-iodo-5-methoxybenzyl)amino)-2-oxoethyl stearate;
2-((4-hydroxy-3-methoxy-benzyl)amino)-2-oxoethyl oleate;
2-((4-hydroxy-2-iodo-5-methoxybenzyl)amino)-2-oxoethyl oleate;
(R,Z)-2-((4-hydroxy-3-methoxybenzyl)amino)-2-oxoethyl 12-hydroxyoctadec-9-enoate;
(R,Z)-2-((4-hydroxy-2-iodo-5-methoxybenzyl)amino)-2-oxoethyl 12-hydroxyoctadec-9-enoate;
(Z)-2-((4-hydroxy-3-methoxy-benzyl)amino)-2-oxoethyl 12-(2-phenylacetoxy)octadec-9-enoate;
(Z)-2-((4-hydroxy-2-iodo-5-methoxybenzyl)amino)-2-oxoethyl 12-(2-phenyl-acetoxy)octadec-9-enoate;
(Z)-2-((4-hydroxy-3-methoxybenzyl)amino)-2-oxoethyl docos-13-enoate;
(Z)-2-((4-hydroxy-2-iodo-5-methoxybenzyl)amino)-2-oxoethyl docos-13-enoate;
(5Z,8Z,11Z,14Z)-2-((4-hydroxy-3-methoxy-benzyl)amino)-2-oxoethyl icosa-5,8,11,14-tetraenoate;
(5Z,8Z,11Z,14Z)-2-((4-hydroxy-2-iodo-5-methoxy-benzyl)amino)-2-oxoethyl icosa-5,8,11,14-tetraenoate;
(4E,8E,12E,16E)-2-((4-hydroxy-3-methoxybenzyl)-amino)-2-oxoethyl 4,8,13,17,21-pentamethyldocosa-4,8,12,16,20-pentaenoate;
(4E,8E,12E,16E)-2-((4-hydroxy-2-iodo-5-methoxy-benzyl)amino)-2-oxoethyl
4,8,13,17,21-pentamethyl-docosa-4,8,12,16,20-pentaenoate;
(E)-2-((3,4-dihydroxybenzyl)-amino)-2-oxoethyl 3,7-dimethylocta-2,6-dienoate;
(E)-2-((4,5-dihydroxy-2-iodo-benzyl)amino)-2-oxoethyl 3,7-dimethylocta-2,6-dienoate;
(4E,8E,12E,16E)-2-((3,4-dihydroxybenzyl)amino)-2-oxoethyl 4,8,13,17,21-pentamethyldocosa-4,8,12,16,20-pentaenoate;
(4E,8E,12E,16E)-2-((4,5-dihydroxy-2-iodobenzyl)-amino)-2-oxoethyl 4,8,13,17,21-pentamethyl-docosa-4,8,12,16,20-pentaenoate;
(E)-2-((3,4-dihydroxy-phenethyl)amino)-2-oxoethyl 3,7-dimethylocta-2,6-dienoate;
(E)-2-((4,5-dihydroxy-2-iodophenethyl)amino)-2-oxoethyl 3,7-dimethylocta-2,6-dienoate;
(4E,8E,12E,16E)-2-((3,4-dihydroxyphenethyl)amino)-2-oxoethyl 4,8,13,17,21-pentamethyldocosa-4,8,12,16,20-pentaenoate;
(4E,8E,12E,16E)-2-((4,5-dihydroxy-2-iodophenethyl)-amino)-2-oxoethyl 4,8,13,17,21-pentamethyl-docosa-4,8,12,16,20-pentaenoate;
2-((3,4-dihydroxyphenethyl)-amino)-2-oxoethyl oleate;
2-((4,5-dihydroxy-2-iodophenethyl)amino)-2-oxoethyl oleate;
(5Z,8Z,11Z,14Z)-2-((3,4-dihydroxyphenethyl)amino)-2-oxoethyl icosa-5,8,11,14-tetraenoate; and
(5Z,8Z,11Z,14Z)-2-((4,5-dihydroxy-2-iodophenethyl)-amino)-2-oxoethyl icosa-5,8,11,14-tetraenoate.

Clause 6. The composition according to any of clauses 1 to 5, wherein the known TRPV1 modulator is 2-((4-hydroxy-2-iodo-5-methoxybenzyl)amino)-2-oxoethyl nonanoate.

Clause 7. The composition according to any of clauses 1 to 3, comprising a synergistic combination of:
a) a known neuronal exocytosis inhibitor which is selected from the group consisting of known peptides derived from SNAP-25 protein, sintaxin protein, synaptobrevin protein (VAMP), Munc protein; and
b) a known TRPV1 modulator as defined in clauses 4 to 6.

Clause 8. The composition according to any of clauses 1 to 7, wherein the known neuronal exocytosis inhibitor is selected from the group consisting of an amino acid sequence deriving from the N-terminal domain of protein SNAP-25, known peptides consisting of an amino acid sequence deriving from the C-terminal domain, and mixtures thereof, wherein protein SNAP-25 is defined by SEQ ID NO 1.

Clause 9. The composition according to clause 8, wherein the known neuronal-exocytosis inhibitor is selected from those which at a concentration of 1 mM inhibit from 15% to 45% of the release of catecholamines in the permeabilized chromaffin cells; where the inhibition of neural-exocytosis is measured by evaluating the strength of said compounds in inhibiting the release of catecholamines induced by calcium in chromaffin cells permeabilized with the detergent digitonin in accordance with the method described in the description.

Clause 10. The composition according to any of clauses 1 to 9, wherein the known neuronal exocytosis inhibitor is selected from the group consisting of
a peptide consisting of an amino acid sequence selected from the amino acid sequence of SEQ. ID NO. 1 or the amino acid sequence of SEQ. ID NO. 2;
the peptides defined by SEQ. ID NO. 1 or SEQ. ID NO. 2 wherein the amino acid at the N-terminus of the peptide is acetylated, and, optionally, wherein the amino acid at the C-terminus of the peptide is aminated;
the peptides defined by SEQ. ID NO. 1 or SEQ. ID NO. 2 wherein the amino acid at the C-terminus of the peptide is aminated, and, optionally, wherein the amino acid at the C-terminus of the peptide is acetylated;
the peptides defined by SEQ. ID NO. 1 or SEQ. ID NO. 2 wherein further contains a reversible chemical modification that increases its bioavailability and facilitates its permeation through the blood brain barrier and epithelial tissue;
a substantially homologous peptide to the peptide defined by SEQ. ID NO. 1 or SEQ. ID NO. 2;
a pharmaceutically or cosmetically acceptable salt of the peptide defined by SEQ. ID NO. 1 or SEQ. ID NO. 2; and
a peptide defined by SEQ. ID NO. 1 or SEQ. ID NO. 2 that has undergone reversible chemical modifications.

Clause 11. The composition according to any of clauses 1 to 10, wherein
a) the known neuronal exocytosis inhibitor is selected from:
   i) a peptide consisting of an amino acid sequence selected from the amino acid sequence of SEQ. ID NO. 1 or the amino acid sequence of SEQ. ID NO. 2;
   ii) the peptides defined by SEQ. ID NO. 1 or SEQ. ID NO. 2 wherein the amino acid at the N-terminus of the peptide is acetylated, and, optionally, wherein the amino acid at the C-terminus of the peptide is aminated; and
   iii) the peptides defined by SEQ. ID NO. 1 or SEQ. ID NO. 2 wherein the amino acid at the C-terminus of the peptide is aminated, and, optionally, wherein the amino acid at the C-terminus of the peptide is acetylated; and
b) the known TRPV1 modulator is 2-((4-hydroxy-2-iodo-5-methoxybenzyl)amino)-2-oxoethyl nonanoate.

Clause 12. The composition according to any of clauses 1 to 11, wherein the known neuronal exocytosis inhibitor is found in a concentration between 0.5% and 0.005% in weight, with regards to the total weight of the composition; and the known TRPV1 modulator is found in a concentration between 0.1% and 0.001% in weight, with regards to the total weight of the composition.

Clause 13. A pharmaceutical composition which comprises a therapeutically effective amount of the composition as defined in any of the clauses 1 to 12 together with one or more pharmaceutically acceptable excipients or carriers.

Clause 14. A pharmaceutical composition as defined in clause 13, for use in the prevention and/or treatment of a disease or condition which courses through excessive neural stimulation of the eccrine gland, wherein the disease or condition is hyperhidrosis.

Clause 15. A known TRPV1 modulator together with one or more pharmaceutically acceptable excipients or carriers, for administration in combination with a known neuronal exocytosis inhibitor, for simultaneous concurrent, or sequential use in the treatment and/or prevention of hyperhidrosis.

Clause 16. The known TRPV1 modulator for administration in combination with a known neuronal exocytosis inhibitor according to clause 15, wherein the known TRPV1 modulator is selected from the group of known TRPV1 agonists capable of desensitizing TRPV1 with an EC50 value≤10 μM, and TRPV1 antagonists capable of inhibiting TRPV1 with an IC50 value≤100 μM; where the activation/inhibition of TRPV1 is measured by calcium microfluorometry assays as described in the description.

Clause 17. known TRPV1 modulator for administration in combination with a known neuronal exocytosis inhibitor according to any of clauses 15 to 16, wherein the known neuronal-exocytosis inhibitor is selected from those which at a concentration of 1 mM inhibit from 15% to 45% of the release of catecholamines in the permeabilized chromaffin cells; where the inhibition of neural-exocytosis is measured by evaluating the strength of said compounds in inhibiting the release of catecholamines induced by calcium in chromaffin cells permeabilized with the detergent digitonin in accordance with the method described in the description.

Clause 18. A cosmetic composition which comprises a cosmetically effective amount of the composition as defined in any of the claims 1 to 12 together with one or more cosmetically acceptable excipients or carriers.

Clause 19. Use of the composition as defined in any of clauses 1 to 12, or the cosmetic composition as defined in clause 18, for the cosmetic, non-therapeutically treatment of perspiration.

Clause 20. A method of cosmetic, non-therapeutic treatment of a subject comprising administering a cosmetically effective amount of a composition according to any one of clauses 1 to 12, or a cosmetic composition according to clause 18, to a subject, wherein the cosmetic, non-therapeutic treatment is the treatment and/or prevention of perspiration.

Clause 21. Use of a cosmetic composition as defined in clause 18, as a skin care agent, wherein the skin care comprises ameliorating the excessive perspiration.

Clause 22. Cosmetic method for the treatment of human perspiration comprising the use of the combination as defined in any of clauses 1 to 12, or the cosmetic composition as defined in clause 18.

1. A composition comprising a synergistic combination of:
a) a known neuronal exocytosis inhibitor; and
b) a known TRPV1 modulator;
wherein the known TRPV1 modulator is selected from the group of known TRPV1 agonists capable of desensitizing TRPV1 with an EC50 value≤10 μM, and TRPV1 antagonists capable of inhibiting TRPV1 with an IC50 value≤100 μM; where the activation/inhibition of TRPV1 is measured by calcium microfluorometry assays as described in the description;
and wherein
the known neuronal-exocytosis inhibitor is selected from those which at a concentration of 1 mM inhibit from 15% to 45[[ ]]% of the release of catecholamines in the permeabilized chromaffin cells, or those which at a concentration of 0.1 mM inhibit from 25% to 50% of the release of α-calcitonin gene-related peptide in intact sensory neurons; where the inhibition of neural-exocytosis is measured by evaluating the strength of said compounds in inhibiting the release of catecholamines induced by calcium in chromaffin cells permeabilized with the detergent digitonin in accordance with the method described in the description; or by enzyme immunoassay evaluating the strength of said compounds in inhibiting the release of α-calcitonin gene-related peptide from intact primary sensory neurons in accordance with the method described in the description.

2. The composition according to claim 1, wherein the known TRPV1 modulator is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts 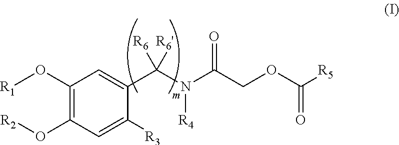
wherein
m is an integer selected from 1 to 3;
R¹, R⁶ and R^{6′} are independently selected from the group consisting of H, optionally substituted C₁-C₈ alkyl, and optionally substituted unsaturated C₂-C₈ hydrocarbon;
wherein the optionally substituted C₁-C₈ alkyl and the optionally substituted unsaturated C₂-C₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶;
R² is H;
R³ is hydrogen or halogen;
R⁴ is selected from the group consisting of H, optionally substituted C₁-C₈ alkyl and optionally substituted unsaturated C₂-C₈ hydrocarbon;
wherein the optionally substituted C₁-C₈ alkyl and the optionally substituted unsaturated C₂-C₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶;
R⁵ is selected from the group consisting of optionally substituted C₃-C₂₈ alkyl, optionally substituted unsaturated C₃-C₂₈ hydrocarbon, and 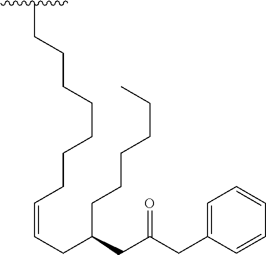 [SMILES: *CCCCCCC/C=C\C[C@@H](CCCCCC)CC(=O)Cc1ccccc1]
wherein the optionally substituted C₃-C₈ alkyl and the optionally substituted unsaturated C₃-C₂₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶.

3. The composition according to claim 1, wherein the known TRPV1 modulator is selected from the group consisting of:
2-((4-hydroxy-3-methoxy-benzyl)amino)-2-oxoethyl butyrate;
2-((4-hydroxy-2-iodo-5-methoxybenzyl)amino)-2-oxoethyl butyrate;
2-((4-hydroxy-3-methoxy-benzyl)amino)-2-oxoethyl pentanoate;
2-((4-hydroxy-2-iodo-5-methoxybenzyl)amino)-2-oxoethyl pentanoate;
2-((4-hydroxy-3-methoxy-benzyl)amino)-2-oxoethyl 3-methylbutanoate;
2-((4-hydroxy-2-iodo-5-methoxybenzyl)amino)-2-oxoethyl 3-methyl-butanoate;
2-((4-hydroxy-3-methoxy-benzyl)amino)-2-oxoethyl hexanoate;
2-((4-hydroxy-2-iodo-5-methoxybenzyl)amino)-2-oxoethyl hexanoate;
(2E,4E)-2-((4-hydroxy-3-methoxybenzyl)amino)-2-oxoethyl hexa-2,4-dienoate;
(2E,4E)-2-((4-hydroxy-2-iodo-5-methoxybenzyl)amino)-2-oxoethyl hexa-2,4-dienoate;
2-((4-hydroxy-3-methoxy-benzyl)amino)-2-oxoethyl heptanoate;
2-((4-hydroxy-2-iodo-5-methoxybenzyl)amino)-2-oxoethyl heptanoate;
2-((4-hydroxy-3-methoxy-benzyl)amino)-2-oxoethyl octanoate;
2-((4-hydroxy-2-iodo-5-methoxybenzyl)amino)-2-oxoethyl octanoate;
2-((4-hydroxy-3-methoxybenzyl)amino)-2-oxoethyl nonanoate;
2-((4-hydroxy-2-iodo-5-methoxybenzyl)amino)-2-oxoethyl nonanoate;
(E)-2-((4-hydroxy-3-methoxy-benzyl)amino)-2-oxoethyl 3,7-dimethylocta-2,6-dienoate;
(E)-2-((4-hydroxy-2-iodo-5-methoxybenzyl)amino)-2-oxoethyl 3,7-dimethylocta-2,6-dienoate;
2-((4-hydroxy-3-methoxy-benzyl)amino)-2-oxoethyl decanoate;
2-((4-hydroxy-2-iodo-5-methoxybenzyl)amino)-2-oxoethyl decanoate;
2-((4-hydroxy-3-methoxy-benzyl)amino)-2-oxoethyl dodecanoate;
2-((4-hydroxy-2-iodo-5-methoxybenzyl)amino)-2-oxoethyl dodecanoate;
2-((4-hydroxy-3-methoxy-benzyl)amino)-2-oxoethyl pentadecanoate;
2-((4-hydroxy-2-iodo-5-methoxybenzyl)amino)-2-oxoethyl pentadecanoate;
2-((4-hydroxy-3-methoxy-benzyl)amino)-2-oxoethyl stearate;
2-((4-hydroxy-2-iodo-5-methoxybenzyl)amino)-2-oxoethyl stearate;
2-((4-hydroxy-3-methoxy-benzyl)amino)-2-oxoethyl oleate;
2-((4-hydroxy-2-iodo-5-methoxybenzyl)amino)-2-oxoethyl oleate;
(R,Z)-2-((4-hydroxy-3-methoxybenzyl)amino)-2-oxoethyl 12-hydroxyoctadec-9-enoate;
(R,Z)-2-((4-hydroxy-2-iodo-5-methoxybenzyl)amino)-2-oxoethyl 12-hydroxyoctadec-9-enoate;
(Z)-2-((4-hydroxy-3-methoxy-benzyl)amino)-2-oxoethyl 12-(2-phenylacetoxy)octadec-9-enoate;
(Z)-2-((4-hydroxy-2-iodo-5-methoxybenzyl)amino)-2-oxoethyl 12-(2-phenyl-acetoxy)octadec-9-enoate;
(Z)-2-((4-hydroxy-3-methoxybenzyl)amino)-2-oxoethyl docos-13-enoate;
(Z)-2-((4-hydroxy-2-iodo-5-methoxybenzyl)amino)-2-oxoethyl docos-13-enoate;
(5Z,8Z,11Z,14Z)-2-((4-hydroxy-3-methoxy-benzyl)amino)-2-oxoethyl icosa-5,8,11,14-tetraenoate;
(5Z,8Z,11Z,14Z)-2-((4-hydroxy-2-iodo-5-methoxy-benzyl)amino)-2-oxoethyl icosa-5,8,11,14-tetraenoate;
(4E,8E,12E,16E)-2-((4-hydroxy-3-methoxybenzyl)-amino)-2-oxoethyl 4,8,13,17,21-pentamethyldocosa-4,8,12,16,20-pentaenoate;
(4E,8E,12E,16E)-2-((4-hydroxy-2-iodo-5-methoxy-benzyl)amino)-2-oxoethyl 4,8,13,17,21-pentamethyl-docosa-4,8,12,16,20-pentaenoate;
(E)-2-((3,4-dihydroxybenzyl)-amino)-2-oxoethyl 3,7-dimethylocta-2,6-dienoate;
(E)-2-((4,5-dihydroxy-2-iodo-benzyl)amino)-2-oxoethyl 3,7-dimethylocta-2,6-dienoate;
(4E,8E,12E,16E)-2-((3,4-dihydroxybenzyl)amino)-2-oxoethyl 4,8,13,17,21-pentamethyldocosa-4,8,12,16,20-pentaenoate;
(4E,8E,12E,16E)-2-((4,5-dihydroxy-2-iodobenzyl)-amino)-2-oxoethyl 4,8,13,17,21-pentamethyl-docosa-4,8,12,16,20-pentaenoate;
(E)-2-((3,4-dihydroxy-phenethyl)amino)-2-oxoethyl 3,7-dimethylocta-2,6-dienoate;
(E)-2-((4,5-dihydroxy-2-iodophenethyl)amino)-2-oxoethyl 3,7-dimethylocta-2,6-dienoate;
(4E,8E,12E,16E)-2-((3,4-dihydroxyphenethyl)amino)-2-oxoethyl 4,8,13,17,21-pentamethyldocosa-4,8,12,16,20-pentaenoate;
(4E,8E,12E,16E)-2-((4,5-dihydroxy-2-iodophenethyl)-amino)-2-oxoethyl 4,8,13,17,21-pentamethyl-docosa-4,8,12,16,20-pentaenoate;
2-((3,4-dihydroxyphenethyl)-amino)-2-oxoethyl oleate;
2-((4,5-dihydroxy-2-iodophenethyl)amino)-2-oxoethyl oleate;
(5Z,8Z,11Z,14Z)-2-((3,4-dihydroxyphenethyl)amino)-2-oxoethyl icosa-5,8,11,14-tetraenoate; and
(5Z,8Z,11Z,14Z)-2-((4,5-dihydroxy-2-iodophenethyl)-amino)-2-oxoethyl icosa-5,8,11,14-tetraenoate.

4. The composition according to claim 1, comprising a synergistic combination of:
a) a known neuronal exocytosis inhibitor which is selected from the group consisting of known peptides derived from SNAP-25 protein, sintaxin protein, synaptobrevin protein (VAMP), Munc protein; and
b) a known TRPV1 modulator,
wherein the known TRPV1 modulator is selected from the group consisting of a compound of formula (I), or a pharmaceutically or cosmetically acceptable salt thereof, or any stereoisomer or mixtures thereof, either of the compound of formula (I) or of any of its pharmaceutically or cosmetically acceptable salts 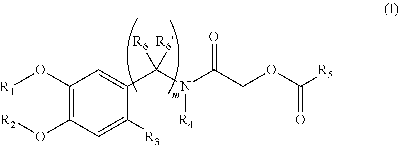
wherein
m is an integer selected from 1 to 3;
R¹, R⁶ and R^{6′} are independently selected from the group consisting of H, optionally substituted C₁-C₈ alkyl, and optionally substituted unsaturated C₂-C₈ hydrocarbon;
wherein the optionally substituted C₁-C₈ alkyl and optionally substituted unsaturated C₂-C₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶;
R² is H;
R³ is hydrogen or halogen;
R⁴ is selected from the group consisting of H, optionally substituted C₁-C₈ alkyl, and optionally substituted unsaturated C₂-C₈ hydrocarbon;
wherein the optionally substituted C₁-C₈ alkyl and the optionally substituted unsaturated C₂-C₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶;
R⁵ is selected from the group consisting of optionally substituted C₃-C₂₈ alkyl, optionally substituted unsaturated C₃-C₂₈ hydrocarbon, and 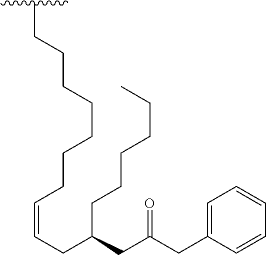 [SMILES: *CCCCCCC/C=C\C[C@@H](CCCCCC)CC(=O)Cc1ccccc1]
wherein the optionally substituted C₃-C₈ alkyl and the optionally substituted unsaturated C₃-C₂₈ hydrocarbon are optionally substituted with one or more substituents selected from the group consisting of
halogen, —COOH, —OH, —NH₂, —COOR⁶, —NO₂, —CF₃, —OCF₃, —CN, —OR⁶, —CONH₂, —CONHR⁶, —NHR⁶, —NHCOR⁶, —NHSO₂R⁶, and —SO₂NHR⁶.

5. The composition according to claim 1, wherein the known neuronal exocytosis inhibitor is selected from the group consisting of an amino acid sequence deriving from the N-terminal domain of protein SNAP-25, known peptides consisting of an amino acid sequence deriving from the C-terminal domain of protein SNAP-25, and mixtures thereof.

6. The composition according to claim 1, wherein the known neuronal exocytosis inhibitor is selected from the group consisting of
a peptide consisting of an amino acid sequence selected from the amino acid sequence of SEQ. ID NO. 1 or the amino acid sequence of SEQ. ID NO. 2;
the peptide defined by SEQ. ID NO. 1 or SEQ. ID NO. 2 wherein the amino acid at the N-terminus of the peptide is acetylated, and, optionally, wherein the amino acid at the C-terminus of the peptide is aminated;
the peptide defined by SEQ. ID NO. 1 or SEQ. ID NO. 2 wherein the amino acid at the C-terminus of the peptide is aminated, and, optionally, wherein the amino acid at the C-terminus of the peptide is acetylated;
the peptide defined by SEQ. ID NO. 1 or SEQ. ID NO. 2 wherein further contains a reversible chemical modification that increases its bioavailability and facilitates its permeation through the blood brain barrier and epithelial tissue;
a substantially homologous peptide to the peptide defined by SEQ. ID NO. 1 or SEQ. ID NO. 2;
a pharmaceutically or cosmetically acceptable salt of the peptide defined by SEQ. ID NO. 1 or SEQ. ID NO. 2; and
a peptide defined by SEQ. ID NO. 1 or SEQ. ID NO. 2 that has undergone reversible chemical modifications.

7. The composition according to claim 1, wherein
a) the known neuronal exocytosis inhibitor is selected from
i) a peptide consisting of an amino acid sequence selected from the amino acid sequence of SEQ. ID NO. 1 or the amino acid sequence of SEQ. ID NO. 2;
ii) the peptide defined by SEQ. ID NO. 1 or SEQ. ID NO. 2 wherein the amino acid at the N-terminus of the peptide is acetylated, and, optionally, wherein the amino acid at the C-terminus of the peptide is aminated; and
iii) the peptide defined by SEQ. ID NO. 1 or SEQ. ID NO. 2 wherein the amino acid at the C-terminus of the peptide is aminated, and, optionally, wherein the amino acid at the C-terminus of the peptide is acetylated;
and
b) the known TRPV1 modulator is 2-((4-hydroxy-2-iodo-5-methoxybenzyl)amino)-2-oxoethyl nonanoate.

8. The composition according to claim 1, wherein the known neuronal exocytosis inhibitor is found in a concentration between 0.5% and 0.005% in weight, with regards to the total weight of the composition; and the known TRPV1 modulator is found in a concentration between 0.1% and 0.001% in weight, with regards to the total weight of the composition.

9. A pharmaceutical composition which comprises a therapeutically effective amount of the composition as defined in claim 1 together with one or more pharmaceutically acceptable excipients or carriers.

10. A method of treatment of a disease or condition from excessive neural stimulation of the eccrine gland, wherein the disease or condition is hyperhidrosis; the method comprising:
administering the pharmaceutical composition as defined in claim 9 to a subject suffering from hyperhidrosis.

11. The method of claim 10, wherein the known neuronal exocytosis inhibitor and the known TRPV1 modulator are administered by simultaneous concurrent use.

12. A cosmetic composition which comprises a cosmetically effective amount of the composition as defined in claim 1 together with one or more cosmetically acceptable excipients or carriers.

13. A method of cosmetic non-therapeutic treatment of perspiration, the method comprising:
administering the cosmetic composition of claim 12 to a subject.

14. The method of claim 10, wherein the known neuronal exocytosis inhibitor and the known TRPV1 modulator are administered by sequential use.

15. A method of skin care, the method comprising:
applying the cosmetic composition as defined in claim 12 to ameliorate excessive perspiration.

16. A cosmetic method for the treatment of human perspiration comprising:
using the composition as defined in claim 1 on a subject.
